(19) United States
(12) Patent Application Publication
(10) Pub. No.: US 2026/0265210 A1
(43) Pub. Date: Sep. 10, 2026

(54) SIMPLIFIED ETHYLENE OXIDE PURIFICATION METHODS

(71) Applicant: Dow Global Technologies LLC, Midland, MI (US)

(72) Inventor: Elias M. Hindy, Missouri City, TX (US)

(21) Appl. No.: 19/573,746

(22) Filed: Mar. 20, 2026

Related U.S. Application Data
(62) Division of application No. 18/692,105, filed on Nov. 15, 2022.

(51) Int. Cl. C07D 301/32; B01D 3/00; B01D 3/14; B01D 3/32; C07C 45/78; C07D 303/04

(52) U.S. Cl. CPC C07D 301/32; B01D 3/007; B01D 3/141; B01D 3/322; C07C 45/78; C07D 303/04

(57) ABSTRACT

The present invention provides simplified, efficient methods for refining a crude ethylene oxide (EO) containing mixture of water, EO, aldehydes and various gases comprising distilling the mixture in a dividing wall column having a receiving side and a product side and drawing each of a purified ethylene oxide stream and an isolated acetaldehyde stream from the product side of the dividing wall column, while minimizing the time during which a mixture containing more than 1 wt. % of each of ethylene oxide and water are in contact at a temperature above 80° C. to 2 minutes or less. No side draw is removed from the dividing wall column on its receiving side. The purification of the crude ethylene oxide needs no stripper or second absorber. The overhead and bottoms from the dividing wall column are recycled into the column or are reused in ethylene oxide production. The resulting purified EO stream may be more than 99.9% pure.

### FIELD

Embodiments relate to methods of refining a crude ethylene oxide feed stream containing water and aldehydes from a manufacturing process comprising distilling the feed stream in a dividing wall column and drawing from the dividing wall column each of a purified ethylene oxide stream and an isolated acetaldehyde stream from the same side of a dividing wall having a receiving side and a product side. More particularly, it relates to a method comprising refining a feed stream of crude ethylene oxide from water, aldehydes, glycols, ethylene, CO₂ and other non-condensables comprising distilling the feed stream in a dividing wall column at, for example, an elevated pressure and drawing from the dividing wall column each of a purified ethylene oxide stream and an isolated acetaldehyde stream from the product side of the dividing wall, while minimizing the time during which a liquid mixture containing more than 1 wt. % of each of ethylene oxide and water are in contact at a temperature above 80° C., for example, to 2 minutes or less.

### INTRODUCTION

Manufactured worldwide in amounts of several million tons per year, ethylene oxide can be prepared in large quantities. For example, catalytic oxidation of ethylene with oxygen over a suitable catalyst, such as a silver-containing catalyst at elevated temperature of, for example, from 100 to 500° C., and at superatmospheric pressure, causes ethylene and oxygen to react and form ethylene oxide. The yields are relatively low, for example, from 5 to 25%; and the reaction product or effluent can include ethylene oxide, unconverted ethylene and oxygen, carbon dioxide, aldehyde impurities, other low molecular weight hydrocarbons, and fixed gases such as argon and nitrogen. The reaction product can be treated, for example, with water via water absorption, steam stripping and reabsorption in water to form a crude ethylene oxide stream that contains lesser amounts of other materials. Crude ethylene oxide may then be further refined to form purified ethylene oxide, i.e having a sufficient purity for industrial applications. However, each of absorption, stripping and readsorption requires a separate column or tower.

The capital required for refining ethylene oxide can be prohibitively costly. Recovery of the ethylene oxide from the gaseous reaction effluent can involve the initial water absorption step, followed by a stripping step, which is in turn followed by reabsorption that may comprise distillation. The absorption of ethylene oxide in water can recover ethylene oxide from unreacted ethylene, oxygen, and/or other gaseous components (e.g., carbon dioxide, nitrogen, argon). In some instances, each part of the refinement process may be carried out in separate distillation columns, which can lead to high equipment costs and, in addition, to safety concerns where areas of concentrated vapor phase or liquid phase ethylene oxide persist. Accordingly, manufacturers often cannot economically justify building or installing small scale units. Further, the maintenance costs for such capital equipment increase with an increase in the number of unit operations required, including multiple pumps and compressor(s). Still further, the refinery processes can prove challenging.

In any heated stream containing ethylene oxide and water, some degree of reaction between ethylene oxide and water to form ethylene glycol can occur. This can lead to further side reactions, such as higher glycol formation or oxidation of the ethylene glycol to form glycol aldehydes. Side reaction activity is highest in systems with higher temperatures and longer residence times.

U.S. Pat. No. 10,035,782 B2 to Olthof discloses methods for recovering an ethylene oxide (EO) from a fat absorbent (FA) feed comprising water, EO in a concentration ranging of from 1 to 15 wt. %, glycols and acetaldehyde comprising passing the FA feed from a loopgas EO absorber to a multi-stage countercurrent distillation zone. The method further comprises obtaining from a left hand side of the distillation zone an acetaldehyde enriched stream, a lean absorbent (LA) stream, a vapor stream enriched in light ends and an EO enriched product stream, and a glycol enriched bottoms stream. Olthof fails to disclose a method wherein an EO enriched product stream is generated without drawing a stream in a form requiring further processing, such as by stripping; further Olthof fails to disclose methods wherein no liquid is separated from the overheads for further processing. Finally, Olthof fails to address any side reaction problem.

The present inventors have sought to reduce the amount of equipment needed to refine ethylene oxide after water absorption without impairing the efficiency and the yield of the refinery.

### SUMMARY

In accordance with the present invention a method of refining a crude ethylene oxide (EO) feed stream containing EO, water and aldehydes from a manufacturing process comprises:
distilling the crude EO feed stream in a dividing wall column containing within it a dividing wall that has a top and a bottom and having a receiving side of the dividing wall and a product side of the dividing wall, and a bottom portion located below the dividing wall; and
drawing out of the dividing wall column each of a purified ethylene oxide stream (purified EO stream) and an isolated acetaldehyde stream (AC stream) from the product side of the dividing wall by drawing the purified EO stream from the dividing wall at a point above a point at which the drawing of the AC stream takes place. The drawing may comprise drawing the AC stream from the product side of the dividing wall at a point 1 to 5 stages (e.g., 1 to 3 stages, etc.) above the bottom of the dividing wall in a dividing wall column that has from 40 to 80 stages; and, further, drawing the purified EO stream from the product side of the dividing wall at the 2^{nd} to 8^{th} stage (e.g., 4^{th} to 6^{th} stage, 2^{nd} to 6^{th} stage, 4^{th} to 8^{th} stage, etc.) from the top of the dividing wall column in a dividing wall column that has from 40 to 80 stages. The method provides a purified EO stream from a crude ethylene oxide feed stream without absorption or stripping. The method provides a purified EO stream that comprises at least 99 wt. %, at least 99.8 wt. %, and/or at least 99.9 wt. % of ethylene oxide, based on the total weight of the purified EO stream.

The distilling may further comprise:
feeding into the dividing wall column water, for example, water at a temperature from 50 to 100° C. and/or from 60 to 95° C., into the receiving side of the dividing wall while feeding the crude EO feed stream into the receiving side of the dividing wall at a point below the point at which the feeding of the water takes place.

The distilling may further comprise:
drawing a bottoms stream comprising water and ethylene glycol from the bottom of the dividing wall column and drawing an overhead stream comprising ethylene oxide, gases and condensable vapors from the top of the dividing wall column. The method may further comprise:
feeding the bottoms stream through a heat exchanger or feed preheater used to preheat the crude EO feed stream before feeding the bottoms stream back to the dividing wall column. More specially, the method may still further comprise:
feeding at least a portion of the bottoms stream through a vent gas scrubber as an absorbant.

The distilling may further comprise:
indirectly heating the dividing wall column in by heating at least a portion of the bottoms stream in two separate reboilers, such as thermosiphon reboilers, wherein a first reboiler is optionally heated by steam and a second reboiler is optionally heated by a recycled vent gas stream from the overhead stream.

Still further, the method comprises:
condensing the overhead stream;
recycling the overhead stream by passing it through a partial condenser, such as a plate and shell heat exchanger, to form a reflux liquid and a gas phase; and

feeding the reflux liquid from the overhead stream back to the dividing wall column. More specifically, the recycling of the overhead stream may comprise:
feeding the overhead stream to a partial condenser, such as a plate and shell heat exchanger, to form a vent gas stream and a reflux liquid stream;
feeding the reflux liquid stream back to the dividing wall column; and
feeding the vent gas stream through a vent gas scrubber; and, then
compressing the vent gas, for example, by either mechanical compressor or by passing it through a two-stage jet compressor comprising, from upstream to downstream, a jet compressor, a condenser, and a second stage jet compressor, to form a recycled vent gas stream before cooling the vent gas in the second reboiler.

The method may further comprise:
preheating the crude ethylene oxide feed stream to a temperature of 90 to 140° C., 90 to 130° C., 100 to 140° C. and/or from 100 to 130° C., using water from a bottoms stream drawn from the dividing wall column, for example, in a heat exchanger that is directly connected to the receiving side of the dividing wall column at the point where the crude EO feed stream enters the dividing wall column. The heat exchanger may comprise a plate and shell heat exchanger. The method may minimize side reactions by limiting the residence time during which a mixture containing more than 1 wt. % of each ethylene oxide and water, based on the total weight of the mixture, is present at a temperature above 80° C., such as, for example, in the preheating and in the dividing wall column, to being 2 minutes or less, 1 minute or less, 45 seconds or less, or 30 seconds or less or 20 seconds or less.

In accordance with exemplary embodiments, the distilling comprises distilling the feed stream in a dividing wall column at, for example, an elevated pressure wherein the pressure at the top of the dividing wall column ranges from 150 KPa absolute to 410 Kpa absolute (1.5 to 4.1 atm absolute).

The crude ethylene oxide (crude EO) feed stream comprises up to 10 wt. % of ethylene oxide and may further comprise water, aldehydes, glycols, ethylene, CO₂ and other non-condensables. The crude EO feed stream may come from an absorber column. The purified EO stream may comprise 99.5 wt. % or more, 99.8 wt. % or more, or 99.9 wt. % or more of ethylene oxide.

### BRIEF DESCRIPTION OF THE DRAWINGS

FIG. 1 depicts an example of a suitable refinery apparatus in accordance with an exemplary embodiment.

FIG. 2 depicts an example of a dividing wall column according to an exemplary embodiment.

### DETAILED DESCRIPTION

In accordance with the methods of the present invention and the apparatus used in such methods, distilling an aqueous crude ethylene oxide (EO) feed stream from water reabsorption of a reaction product of ethylene oxide formation results in an ethylene oxide stream which is equally pure or purer than that resulting from known ethylene oxide refining operations while using substantially less equipment. The present inventor has surprisingly discovered a method of distilling the crude EO feed stream that is equally or more efficient than known prior art methods, even though the methods may comprise distilling at a higher temperature and pressure than known methods and comprises fewer refining steps. In particular, the methods enable removal of pure ethylene oxide product from the right or downstream product side of a dividing wall column and removal of an enriched by-product stream including acetaldehyde stream, or isolated acetaldehyde, without removing any other liquid from the product side of the column and without the need for further refinement of the purified ethylene oxide stream. The present invention may minimize the number of pumps, distillation columns and heat exchangers needed to process all of the various feeds and streams. Further, the method may remove at least a stripper column and an absorber column from the refining of the product of any reaction used to make ethylene oxide. For example, the method enables reabsorbing in water the reaction product of ethylene and oxygen. In addition, the methods use partial condensers and thereby enable reduced fluid hold-up in any of the apparati used, thereby reducing the residence time needed to produce a refined ethylene oxide stream. Accordingly, the methods minimize residence time of ethylene oxide and water, at a temperature of 80° C. or higher, thereby reducing opportunities for side reactions that lower reaction yield, reduce refinery efficiency, and which can cause fouling and create hazardous conditions in a reactor. The methods thereby avoid the need for ethylene glycol removal from the bottoms stream or for formaldehyde treatment, removal or bleed from the overhead stream. As the methods and apparati required are simpler than in known ethylene oxide refinery operations, the methods enable a reduced capital footprint and use less energy. For example, the methods and the apparati at certain operating conditions may eliminate the need for chilled water for condensing.

The singular forms “a,” “an,” and “the” include plural referents unless the context clearly dictates otherwise. Unless defined otherwise, the terms used herein have the same meaning as is commonly understood by one skilled in the art.

Unless otherwise indicated, any term containing parentheses refers, alternatively, to the whole term as if no parentheses were present and the same term without that contained in the parentheses, and combinations of each alternative. Thus, the term “(acet)aldehyde” encompasses, in the alternative, an aldehyde, or acetaldehyde, or mixtures thereof.

The endpoints of all ranges directed to the same component or property are inclusive of the endpoint and are independently combinable.

Unless otherwise indicated, conditions of temperature and pressure are room temperature (23° C.) and standard pressure (101.3 kPa), also referred to as “ambient conditions”. And, unless otherwise indicated, all conditions include a relative humidity (RH) of 40%.

As used herein the term “aqueous” means that the continuous phase or medium is water and from 0 to 49.9 wt. %, based on the weight of the medium, of water-miscible compound(s) or molecules. Preferably, “aqueous” means water.

As used herein, the term “ASTM” refers to publications of ASTM International, West Conshohocken, PA.

As used herein, the term “column” refers to, for example, a generally upright, cylindrical column or tower containing separation stages, such as trays, for example, sieve trays or cross-flow trays, and/or packing elements, wherein the trays and/or packing elements provide a surface area for a liquid and a gas to come into contact, facilitating mass transfer between the liquid and the gas. As used herein, the term “distilling” or “distillation” refer to a process of separating compounds based on differences in their volatility by vaporizing them and, subsequently, condensing them, for example, for purification or concentration. Distillation can be performed on an aqueous mixture to purify, recover, and/or separate ethylene oxide, where the “aqueous mixture” may be a mixture of ethylene oxide, water, and other compounds in liquid form. As used herein, the terms “distill,” “recover,” “purify,” and “separate” should be understood to refer to distilling as it is described herein.

As used herein, the term “separation stage” means a volume, device or combination of devices in a column, tower or other distillation apparatus within or at which phases are brought into intimate contact so that mass transfer occurs between the phases tending to bring them to equilibrium, and where the phases can then be mechanically separated. Each tray of a tray tower or column and/or packing of a packed tower or column having a height equivalent to a theoretical plate (“HETP”) is a separation stage or location where fluids are brought into intimate contact, interphase diffusion occurs, and the fluids are separated. As such, the number of trays in a distillation apparatus can also be attributed to an equivalent number of separation stages that are obtained by using packing. Thus, unless otherwise defined, the terms separation stage, tray and/or packing having one HETP can be used interchangeably.

As used herein, the term “wt. %” means weight %.

The method may simplify the distillation of crude ethylene oxide by eliminating at least two sidestreams and their respective recycling loops, thereby eliminating several pumps, and at least one condensing or heat exchanger apparatus. The methods can be used to purify the crude ethylene oxide feed stream. The crude ethylene oxide feed stream may be produced by reabsorption of ethylene oxide gas with water and may comprise water, ethylene oxide (EO), ethylene glycol, oligo(ethylene glycol)s, aldehydes, such as formaldehyde and/or acetaldehyde, carbon dioxide, and methane. The methods may be thought of as distilling the crude EO feed stream using the apparati of the present invention. The crude EO feed stream may be formed, as follows:

Ethylene oxide can be separated and recovered from a gaseous reaction effluent by reabsorbing the ethylene oxide reaction product in water. For example, the gaseous reaction effluent from a reactor can be scrubbed in an absorber column, such as with water, to form an aqueous mixture containing ethylene oxide and a lesser amount of impurities. The present invention may eliminate the need to pass any reaction product to a stripper, such as a steam stripper, to a second ethylene oxide absorber. From mere reabsorption, an aqueous mixture which is the crude EO feed stream can be obtained.

The method of the present invention comprises:
distilling a crude EO feed stream in a dividing wall column, such as a dividing wall column containing within it a dividing wall having a receiving side of the dividing wall and a product side of the dividing wall; and
drawing out of the dividing wall column each of a purified ethylene oxide stream (purified EO stream) and an isolated acetaldehyde stream (AC stream) from the same side of the dividing wall, wherein the point at which drawing of the purified ethylene oxide stream occurs above the point at which drawing of the AC stream occurs. Preferably, the method comprises drawing the purified EO stream from the product side of the dividing wall at the 2^{nd} to 8^{th} stage or tray from the top of the dividing wall column and, further, comprises drawing the AC stream from the product side of the dividing wall at a point 1 to 5 stages or trays above the bottom of the dividing wall.

The dividing wall column further comprises two reboilers, which are preferably thermosiphon reboilers. Accordingly, the distilling of the present invention further comprises:
indirectly heating the dividing wall column by heating at least a portion of the bottoms stream in two separate reboilers, such as a first and a second reboiler, wherein the second reboiler is optionally heated by recycled vent gas. Further, heating the first reboiler may comprise heating it by steam.

Further, the distilling may comprise:
feeding into the dividing wall column water, for example, water at a temperature of from 50 to 100° C., or, preferably, from 60 to 95° C., into the receiving side of the dividing wall while feeding the crude EO feed stream into the receiving side of the dividing wall column at a point below a point at which the feeding of the water takes place. The water feed is warm and can range higher than 100° C. owing to the increased pressure in the column.

The method comprises distilling at pressures, such as from 1.5 to 4.1 atmospheres absolute (e.g., 3 to 4.1 atmospheres absolute), as determined at the top of the dividing wall column. Preferably, the pressure in the dividing wall column is adjusted by a pressure control valve located above a vent gas scrubber used to recover vent gases from the overhead stream.

To minimize side reactions, such as ethylene glycol or glyol ether formation, the method of the present invention comprises distilling so that the residence time of heated water and ethylene oxide together at more than 80° C. remains less than 2 minutes or less than 45 seconds or less than 30 seconds. The design and location of the feed preheater and the feeding of the crude EO feed stream into the receiving side of the dividing wall column impact the residence time, for example, by locating the feed preheater in direct connection to the receiving side of the dividing wall column. The short residence time for combined heated water and ethylene oxide (EO) includes both a short distance from feed preheater to column and few trays and/or stages in the column containing “substantial amounts” of both water and EO. As used herein, the term “substantial amount” means 1 wt. % or, preferably, 0.5 wt. % or more of a given component in a fluid mixture. Accordingly, in the method of the present invention the residence time in question preferably means the time during which 1 wt. % or more of each of ethylene oxide and water are in contact at a temperature above 80° C.

The method of the present invention further comprises:
condensing the overhead stream, such as in a partial condenser, for example, a plate and shell condenser, to form a vent gas stream and a reflux liquid; and,
feeding the reflux liquid back to the dividing wall column to recycle the overhead stream.

The condensing of the overhead stream forms a vent gas stream and a reflux liquid which is returned to the top of the dividing wall column. Further, because the methods avoid side reactions and because they isolate an acetaldehyde stream, the bottoms stream comprises mostly, if not nearly all, water. Accordingly, the bottoms stream may be used as heating water, such as in preheating the crude EO feed stream.

The recycling of the overhead stream comprises passing the vent gas stream through a water scrubber, feeding the scrubbed vent gas stream through a compressor, for example, either a mechanical compressor or a two-stage jet compressor comprising, from upstream to downstream, a jet compressor, a condenser and a jet compressor, before further condensing the vent gas in the second reboiler. If used, the jet compressors each incorporate steam at a pressure higher than the inlet vent gas stream and thereby create a steam containing vent gas stream which may be used to heat the recycled bottoms stream in the second reboiler. Thus, in such an embodiment, the second reboiler recovers steam from the recycled vent gas.

The feed preheater in the apparatus of the present invention may comprise a plate and shell heat exchanger rather than, for example, a shell and tube heat exchanger. Using a lower volume condenser or heat exchanger minimizes residence time at higher temperatures and minimizes hold up time in the methods of the present invention.

In accordance with the methods of the present invention, the amount (by weight) of ethylene oxide in the purified ethylene oxide stream may be at least 99 wt. %, or, preferably, at least 99.5 wt. %, or still more preferably at least 99.8 wt. %.

In accordance with the methods of the present invention, the amount of acetaldehyde in the purified ethylene oxide stream is no more than 30 ppm, or, preferably, no more than 20 ppm, or, more preferably no more than 10 ppm. The isolated acetaldehyde stream comprises nearly all, or at least 85 wt. % of all acetaldehyde from the crude EO feed stream.

The distilling in the dividing wall column may include a wide range of temperatures. As used herein, the term “temperature” or “reference temperature” in the distilling of the present invention refers to the temperature of the dividing wall column at the point where the crude EO feed stream enters the column. In practice, the temperatures in the column can vary significantly during operation. For example, a temperature gradient may exist within the dividing wall column, with the highest temperature in the lower section where liquids are volatilized or stripped and the lowest temperature in the reabsorption region, where gases may be reabsorbed into liquids or condensed to form reflux. This gradient may be a gradual change across the column and/or various sections of the column, or may be an abrupt temperature change. The operating temperature of the distillation section can range from 20 to 150° C. The operating temperature of the stripping section of the column comprising the lower portion of the dividing wall can range from 100 to 150° C. Peak temperatures in reboilers generally range from 135 to 150° C. and are higher under higher pressure to facilitate boiling or offgassing.

The present invention avoids the need for refining the crude EO feed stream to remove glycols or glycol ethers. In ethylene oxide production, ethylene oxide recovery operations may be operated along with downstream product manufacturing units, such as, for example an ethylene glycol manufacturing unit. Thus, the ethylene oxide processes can also be interlinked with ethylene glycol manufacturing processes. However, in the method of the present invention, a bottoms stream from the dividing wall column only builds up ethylene glycol slowly.

Periodically, a bottoms stream comprising water and a build up of ethylene glycol may be routed out of a bottoms recycle loop by a bottoms effluent control valve, and sent downstream and through a distillation column to be further refined.

In operation of the dividing wall column, the crude ethylene oxide feed stream enters a lower portion of the receiving side of the column, and, optionally, a condensate (water) stream is fed to the column above the preheated crude EO feed stream. The preheated crude EO feed stream contains water and ethylene oxide and rapidly separates into EO containing vapor and denser liquid, which is mostly water, upon entry into the column. The contact between downward falling liquid water and upward rising vapor at the packing and/or trays inside of the column separates the carbon dioxide, water, and aldehydes from ethylene oxide. The overhead vapor stream, which contains light impurities, such as carbon dioxide, oxygen, nitrogen, and argon, among others, is partially condensed in a condenser to yield a vent gas and a liquid reflux stream. The vent gas is scrubbed to remove any remaining ethylene oxide which is recycled to the dividing wall column, while the remaining vent gas is compressed and ultimately recycled to an ethylene oxide forming reactor. Heavier components from the overheads, as liquids, are condensed and are sent back to the column via a reflux stream. The bottoms from the dividing wall column are split and fed to a reboiler, such as a thermosiphon reboiler, that reheats part of the bottoms stream and vaporizes a portion thereof which is then fed back into the column. A second reboiler uses heat from recycled steam, to vaporize part of the bottoms stream which is then fed back into the column.

The surprising improvements in the method amount to more than just selecting design and process control parameters. However, as shown in U.S. patent publication 2017/0033571 A1 to Idowu et al., process variables can further be adjusted in process by determining the concentration of one or more impurities in an ethylene oxide purification column stream having at least one impurity by injecting a sample volume from a liquid product side stream into a gas chromatograph train having a gas chromatograph effluent stream; and generating at least one signal indicative of the corresponding concentration of the at least one impurity. A mathematical model relating a predicted concentration of the at least one impurity in the product stream to selected ethylene oxide purification column process variables can be updated based on the at least one signal; and an updated predicted concentration of the at least one impurity can be calculated therefrom. Then at least one column process variable can be adjusted based on the updated predicted concentration of the at least one impurity.

The apparatus of the present invention comprises a dividing wall column and its associated feeds and streams. The dividing wall column of the present invention includes a dividing wall having a receiving side and a product side, an optional water feed on the receiving side of the dividing wall, which may be the left-hand side of the dividing wall, a feed for a crude EO feed stream located below the optional water feed, a bottoms outlet, an overhead outlet and, located on the product side of the dividing wall, product side draws for each of the purified ethylene oxide stream and an isolated acetaldehyde stream. In addition to the dividing wall column, the apparatus of the present invention comprises two reboilers and recycle loops for each of the overhead stream and the bottoms stream—the two reboilers being used to heat the bottoms stream and thereby heat the dividing wall column—a feed preheater, and a bottoms pump in the bottoms stream. Thus, the apparatus further comprises each of a vent gas scrubber, and an overhead condenser. The overhead stream recycle loop comprises one or more compressors to process vent gas before it is recycled. Finally, the apparatus comprises a pressure control valve located above the vent gas scrubber.

The dividing wall column of the present invention may be a vertically oriented cylindrical column made of stainless steel. As used herein, the term “dividing wall column” refers to a fractional distillation column comprising a substantially fluid tight vertical wall extending through a substantial portion of its height and located inside the column, e.g., in and/or near a central portion thereof, thereby dividing this section of the column into at least two vertical, parallel vapor-liquid contacting sections, as well as a bottom section below the dividing wall; the first vertical parallel section may be termed the “receiving side” of the column; and the second vertical parallel section may be termed the “product side”. The first vertical parallel section may be the left-hand side. The top and bottom of the wall terminate in the column at a point distant from the respective ends of the column, thereby allowing open communication across the column both above and below the dividing wall. Each section contains fractionation trays and/or packing to promote separation. Larger full width or full diameter trays and/or packing are located in the bottom portion of the column which is located below the dividing wall. Larger full width or full diameter trays and/or packing may be beneficial to be located in the top portion of the column, which is located above the dividing wall. Distributor trays at the top of the column or below the rectification section, if present, may serve to feed reflux back individually into each dividing wall section of the column. In operation, generally, a feed stream enters the receiving side, or on the receiving side of the dividing wall section of the column. Further, in a dividing wall column, the product is divided into product streams, with two streams removed from the product side of the column opposite the receiving side. The other two product streams comprise the overhead and the bottom streams, as in a conventional column. Dividing wall columns may operate in conventional fractional distillation conditions. The operating conditions within a dividing wall column may be adjusted according to processing conditions. For example, a column may be operated at a wide range of pressures, ranging from sub-atmospheric (i.e., vacuum), to near atmospheric, to super atmospheric. A dividing wall column may be configured to be of a variety of shapes and orientations. The dividing wall column may be of a variety of sizes. For example, a dividing wall column having a cylindrical shape can have a diameter ranging from 60 cm to 8 m, or 5.5 m or more, or 6 m or more, or up to 8 m, and a height ranging from 6 to 75 m, or 65 m or more. Further, the dividing wall column may be made of any material commonly used in making such columns, for example, stainless steel. Preferably, structured packing is used for column separation to minimize column hold-up time and may reduce column temperatures by reducing column pressure drop, and thus minimize glycol by-product formation. Depending on the material balance and equilibrium considerations of the substances, for example, ethylene oxide, water, and other compounds in liquid form, to be separated, one skilled in the art can calculate the number of equilibrium stages or theoretical trays for use in a given distillation apparatus. One can determine the efficiency of each separation stage, and thereby determine the actual number of separation stages (such as trays or packing height) used by the mechanical design and the condition of operation for a given distillation apparatus. Accordingly, in the methods of the present invention, the number of equilibrium stages may substitute for the number of separation stages or trays as disclosed herein by using the efficiency of each separation stage of the instantly disclosed dividing wall column.

The condensers in the apparatus of the present invention comprise partial condensers, which are heat exchangers suitable to treat recycle streams. The condenser in the overhead stream recycle loop splits the overhead stream into vent gas and a reflux liquid stream. The vent gas recycle loop comprises a scrubber and a compressor system.

The dividing wall column has two reboilers. The first reboiler may comprise a thermosiphon reboiler using utility steam to heat the bottoms stream. The second reboiler may also comprise a thermosiphon reboiler using recycle steam, to simultaneously provide heat to the dividing wall column and to separate any condensables from the recycled vent gas stream.

The feed preheater of the present invention comprises a heat exchanger designed to minimize the residence time of ethylene oxide (EO) mixed with water in the receiving side of the dividing wall column and, thus, reduce glycol by-product formation. The heat exchanger may comprise a plate containing the feed and a shell preferably comprising a recycled bottoms stream from the column. By heating the ethylene oxide feed stream prior to its entry into the dividing wall column, less energy is spent heating the feed stream inside the dividing wall column. The apparatus and method of the present invention maximize the energy input into the feed preheater or heat exchanger, such as via the recycling of various streams or sources of heat. The boil-up in the dividing wall column is a combination of vapor for reflux, vapor for ethylene oxide to be removed, e.g., as a liquid side stream at a side take-off, and the vapor of vent gases to be removed at the top of dividing wall column, as discussed below.

The present inventor has found that a small band of acetaldehyde exists in the product side (e.g., right hand) of the dividing wall column below the purified ethylene oxide stream product draw and has located an acetaldehyde purge or draw at that point.

In the dividing wall column, the reflux liquid from the condenser is returned to the column. Any formaldehyde present in the column mostly goes out the bottoms, with a small portion leaving with the purified EO stream. Vapor from the condenser is sent to a vent scrubber and any resulting vent gases may generally be non-condensibles. The vent gas from the scrubber is recycled to the process through compression. All liquid from the vent scrubber is returned to the column.

Any plate and shell heat exchanger can be operated using counter-flow, with an absorbant, such as water entering the heat exchanger at a high temperature at the bottom of the heat exchanger. As the absorbant flow heats a heat exchanger feed stream, energy is transferred from the fluid to the feed stream, in effect cooling the fluid. The cooled absorbant fluid can exit the heat exchanger from the side of the heat exchanger or at the top. The heat exchanger can also be operated using a parallel flow, preferably where the exchanger is disposed horizontally.

In the Figures herein, as will be appreciated, referenced features shown can be added, exchanged, and/or eliminated so as to provide any number of additional variants of the methods and/or apparati. In addition, the proportion and the relative scale of the elements provided in the figures are intended to illustrate an example of the present invention, and should not be taken in a limiting sense.

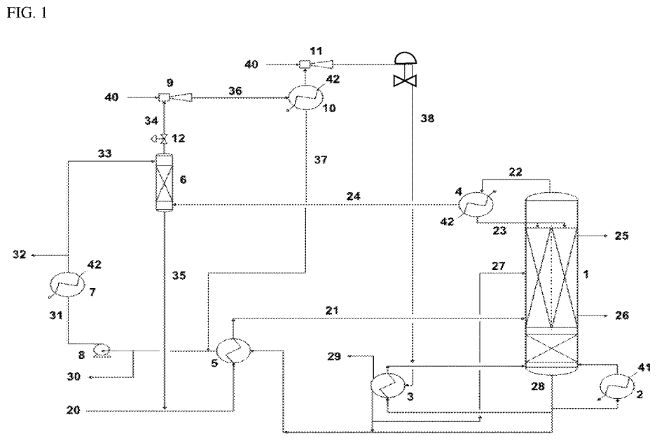

As shown in FIG. 1, the apparatus depicted comprises dividing wall column (1), the associated feeds therefore and outlets or draws therefrom, and the streams and refining equipment associated with the feeds and outlets. Crude ethylene oxide (20) liquid is fed to dividing wall column (1) as dividing wall column feed (21) after flowing it through feed preheater (5). Warm or hot water (27) is fed into the column at a point located above the ethylene oxide feed (21). The receiving side of the dividing wall (not numbered, and represented only as a dotted line), shown as its left-hand side, separates CO₂, ethylene oxide, and non-condensibles from the heavier water and ethylene glycol. The product purified ethylene oxide stream (25) is taken as a liquid side draw from the product side of the dividing wall (shown as the right hand side) at the 2^{nd} to 8^{th} stage (stages not shown) from the top of the column. An isolated acetaldehyde stream (26) as a liquid side draw is taken at from 1 to 5 stages above the bottom of the dividing wall in the product side of the column. Water and heavy by-products are removed out the column bottoms (28). Further, column overheads (22) are fed to partial condenser (4) that is cooled with water (42) and the liquid phase becomes column reflux (23) which is fed back to both sides, the receiving side and the product side, of the dividing wall. Column bottoms (28) stream runs through feed preheater (5), a cross exchanger, to preheat column feed (20) before running through a recycle pump (8) in the form of a bottoms recycle stream (31), which is then fed to bottoms recycle cooler (7), another exchanger cooled with water (42), to form a stream that is nearly all water and which is partly used as vent scrubber water feed (33). Not shown, a bottoms effluent control valve in bottoms recycle stream (31) above bottoms recycle pump (8) allows the drawing off of bottoms effluent (30). Another portion of bottoms (28) is fed into each of thermosiphon reboiler (2) heated by steam (41) and second reboiler (3) heated by exhaust steam (38). From overhead stream (22), overhead vapor (24) from partial condenser (4) is fed to vent gas scrubber (6) where ethylene oxide is scrubbed with cooled dividing wall column bottoms (33), and the bottoms from vent gas scrubber (35), which include the condensed liquid from the dividing wall column bottom, are recycled back as liquid bottoms from the vent gas scrubber into the column feed stream via crude ethylene oxide (20) which is sent through the feed preheater (5). Overheads of recovered vent gas (34) from vent scrubber (6) are compressed using jet compressors (9) and (11), respectively, in two stages, with each jet compressor stage having an inlet for high pressure steam (40) and thereby form jet compressor condensate (37) from condenser (10) and exhaust steam (38) from second stage jet compressor (11). Water (42) cooled condenser (10), located between jet compressors (9) and (11) creates compressed vent gas which is used to heat second column reboiler (3); and the non-condensables from vent gas (29) are recycled for recovery of ethylene in an upstream process. Condensate (37) from condenser (10) is recycled into the column feed stream via crude ethylene oxide (20) which is sent through the feed preheater (5). Pressure in the refinery is controlled by pressure control valve (12) located above vent gas scrubber (6).

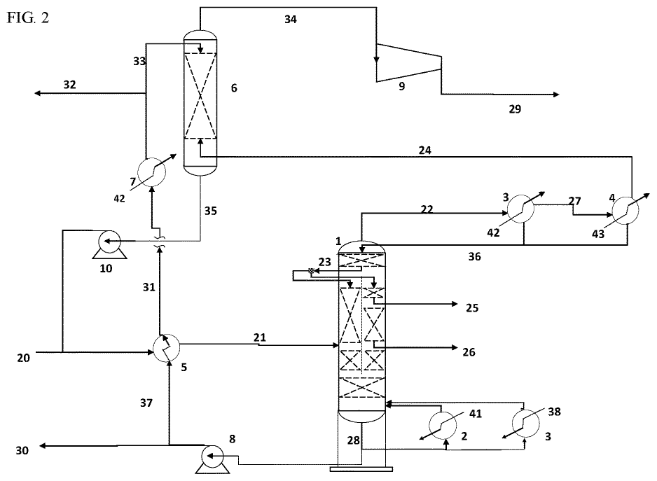

As shown in FIG. 2, the apparatus depicted comprises dividing wall column (1), the associated feeds therefore and outlets or draws therefrom, and the streams and refining equipment associated with the feeds and outlets. Crude ethylene oxide (20) liquid is fed to dividing wall column (1) as dividing wall column feed (21) after flowing it through feed preheater (5). The receiving side of the dividing wall (not numbered, and represented only as a dotted line), shown as its left-hand side, separates CO₂, ethylene oxide, and non-condensibles from the heavier water and ethylene glycol. The product purified ethylene oxide stream (25) is taken as a liquid side draw from the product side of the dividing wall (shown as the right hand side) at the 2^{nd} to 8^{th} stage (stages not shown) from the top of the column. An isolated acetaldehyde stream (26) as a liquid side draw is taken at from 1 to 5 stages above the bottom of the dividing wall in the product side of the column. Water and heavy by-products are removed out the column bottoms (28). Further, column overheads (22) are fed to partial condenser(s) (3 & 4) that are cooled with water (42) and/or chilled water (43), depending on the operating pressure of the column, and the liquid phase becomes column reflux (36 or 23, depending on if there is a rectification section) which is fed back to either the rectification section or to both sides, the receiving side and the product side, of the dividing wall. Column bottoms (28) stream runs through a recycle pump (8) and then a feed preheater (5), a cross exchanger, to preheat column feed (20). The bottoms recycle stream (31), is then fed to bottoms recycle cooler (7), another exchanger cooled with water (42), to form a stream that is nearly all water and which is partly used as vent scrubber water feed (33). Not shown, a bottoms effluent control valve in bottoms recycle stream (37) above bottoms recycle pump (8) allows the drawing off of bottoms effluent (30). Another portion of bottoms (28) is fed into each of thermosiphon reboiler (2) heated by steam (41) and second reboiler (3) heated by recycle steam (38) that may be from another part of the process. From overhead stream (27), overhead vapor (24) from partial condenser (4) is fed to vent gas scrubber (6) where ethylene oxide is scrubbed with cooled dividing wall column bottoms (33) and the bottoms from vent gas scrubber (35) are recycled back into the column feed stream via crude ethylene oxide (20) which is sent through the feed preheater (5). Overheads of recovered vent gas (34) from vent scrubber (6) are compressed using a mechanical compressor (9). The non-condensables from vent gas (29) are recycled for recovery of ethylene in an upstream process. Pressure in the refinery is controlled by pressure control valve (12) located above vent gas scrubber (6).

### EXAMPLES

The following examples illustrate the present invention. Notwithstanding that the numerical ranges and parameters setting forth the broad scope of the invention are approximations, numerical values set forth in the specific examples are reported as precisely as possible. Any numerical value inherently contains certain errors necessarily resulting from the standard variation found in their respective testing measurements.

Simulation of Distillation method using apparatus shown in FIG. 1: A refinery as shown in FIG. 1 having a dividing wall column with 58 stages and a dividing wall extending from stages 1 to 50 (from the top) was evaluated using a process simulation tool. The refinery comprised a crude ethylene oxide (EO) feed (21) location between stages 48 and 49 (from the top), a product side purified EO stream draw (25) at stage 5 (from the top), an acetaldehyde stream draw (26) at stage 48 (from the top). The residence time in the feed preheater (5) was estimated to be 16 seconds and the residence time in stages 47-52 (from the top) which includes the top 2 stages in the bottom section of the column and the bottom 4 stages in the receiving side of the divided wall section of the column was estimated to be 30 seconds. The temperatures for these stages where EO composition is greater than 1 wt % are estimated to be between 111-135° C. The dividing wall column (1) was simulated with an operating pressure at the top of the column of 3.6 bar absolute (~360 KPa absolute). The crude ethylene oxide feed in the method, corresponding to (20) in FIG. 1, comprised approximately 2.32 wt. % of ethylene oxide, 0.05 wt. % of carbon dioxide, 2 ppm of acetaldehyde, 2.22 wt. % of ethylene glycol, 44 ppm of formaldehyde, and 95.34 wt. % of water.

The resulting purified EO stream (stream (25) in FIG. 1) comprised approximately 99.998 wt. % of ethylene oxide, 0.02 ppm of water, 11 ppm of acetaldehyde, 4 ppm of formaldehyde, and 0.2 ppm of carbon dioxide. The isolated acetaldehyde stream (stream (26) in FIG. 1) comprised approximately 8.04 wt. % of ethylene oxide, 1.86 wt. % of acetaldehyde, 0.02 wt. % of formaldehyde, and 90.08 wt. % of water.

The yield of the refinery in purified EO stream (25) was estimated to be 99.10% of ethylene oxide recovered, based on the total weight of all ethylene oxide present in the crude ethylene oxide (20) liquid. The energy input into the simulated distillation was estimated to be 2.15 mass units (e.g Kg) of steam input per recovered mass unit of EO.

As is clearly shown in the simulation results, the method yielded an ethylene oxide stream of very high purity with energy input only from steam used to heat the first reboiler and, indirectly, to heat the second reboiler. Further, the resulting products comprised no ethylene glycol.

### Mid Pressure

Simulation of Distillation method using apparatus shown in FIG. 2: A refinery as shown in FIG. 2 having a dividing wall column with 64 stages and a dividing wall extending from stages 1 to 51 (from the top) was evaluated using a process simulation tool. The refinery comprised a crude ethylene oxide (EO) feed (21) location between stages 47 and 48 (from the top), a product side purified EO stream draw (25) at stage 4 (from the top), an acetaldehyde stream draw (26) at stage 49 (from the top). The dividing wall column (1) was simulated with an operating pressure at the top of the column of ~334 kPa absolute. The crude ethylene oxide feed in the method, corresponding to (20) in FIG. 2, comprised approximately 83 ppm inerts, 0.056 wt % methane/ethylene, 2.36 wt. % of ethylene oxide, 0.014 wt. % of carbon dioxide, 2 ppm of acetaldehyde, 2.37 wt. % of ethylene glycol, 0.08 wt % higher glycols, 13 ppm of formaldehyde, and 95.11 wt. % of water.

The resulting purified EO stream (stream (25) in FIG. 2) comprised approximately 99.9994 wt. % of ethylene oxide, 5 ppm of acetaldehyde, 0.4 ppm of formaldehyde, and 1 ppm of carbon dioxide. The isolated acetaldehyde stream (stream (26) in FIG. 2) comprised approximately 97.52 wt. % of ethylene oxide, 1.9 wt. % of acetaldehyde, and 0.58 wt. % of water.

The yield of the refinery in purified EO stream (25) was estimated to be 94.5% of ethylene oxide recovered, based on the total weight of all ethylene oxide present in the crude ethylene oxide (20) liquid. The energy input into the simulated distillation was estimated to be 2.56 mass units (e.g Kg) of steam input per recovered mass unit of EO.

As is clearly shown in the simulation results, the method yielded an ethylene oxide stream of very high purity with energy input only from steam used to heat the first reboiler and, indirectly, to heat the second reboiler. Further, the resulting products comprised no ethylene glycol.

### Low Pressure

Simulation of Distillation method using apparatus shown in FIG. 2: A refinery as shown in FIG. 2 having a dividing wall column with 64 stages and a dividing wall extending from stages 1 to 51 (from the top) was evaluated using a process simulation tool. The refinery comprised a crude ethylene oxide (EO) feed (21) location between stages 47 and 48 (from the top), a product side purified EO stream draw (25) at stage 4 (from the top), an acetaldehyde stream draw (26) at stage 49 (from the top). The dividing wall column (1) was simulated with an operating pressure at the top of the column of ~164 kPa absolute. The crude ethylene oxide feed in the method, corresponding to (20) in FIG. 2, comprised approximately 83 ppm inerts, 0.056 wt % methane/ethylene, 2.36 wt. % of ethylene oxide, 0.014 wt. % of carbon dioxide, 3 ppm of acetaldehyde, 2.45 wt. % of ethylene glycol, 0.08 wt % higher glycols, 56 ppm of formaldehyde, and 95.02 wt. % of water.

The resulting purified EO stream (stream (25) in FIG. 2) comprised approximately 99.9995 wt. % of ethylene oxide, 4 ppm of acetaldehyde, 0.4 ppm of formaldehyde, and 0.3 ppm of carbon dioxide. The isolated acetaldehyde stream (stream (26) in FIG. 2) comprised approximately 97.74 wt. % of ethylene oxide, 1.67 wt. % of acetaldehyde, and 0.59 wt. % of water.

The yield of the refinery in purified EO stream (25) was estimated to be 97.4% of ethylene oxide recovered, based on the total weight of all ethylene oxide present in the crude ethylene oxide (20) liquid. The energy input into the simulated distillation was estimated to be 2.09 mass units (e.g Kg) of steam input per recovered mass unit of EO.

As is clearly shown in the simulation results, the method yielded an ethylene oxide stream of very high purity with energy input only from steam used to heat the first reboiler and, indirectly, to heat the second reboiler. Further, the resulting products comprised no ethylene glycol.

I claim:

1. A method of refining a crude ethylene oxide (EO) feed stream containing EO, water and aldehydes from a manufacturing process, the method of refining comprising:
distilling the crude EO feed stream in a dividing wall column containing within it a dividing wall having a top and a bottom and having a receiving side of the dividing wall and a product side of the dividing wall, and a bottom portion located below the dividing wall; and
drawing from the dividing wall column each of a purified ethylene oxide stream (purified EO stream) and an isolated acetaldehyde stream (AC stream) from the product side of the dividing wall by drawing the purified EO stream from the dividing wall column at a point above a point at which the drawing of the AC stream takes place, thereby providing a purified EO stream that comprises at least 99.5 wt. % of ethylene oxide, based on the total weight of the purified EO stream, wherein:
the drawing includes:
drawing the AC stream from the product side of the dividing wall at a point 1 to 5 stages above the bottom of the dividing wall, the dividing wall column having from 40 to 80 stages, and
drawing the purified EO stream from the product side of the dividing wall at the 2^{nd} to 8^{th} stage from the top of the dividing wall; and
the pressure at a top of the dividing wall column ranges from 150 KPa absolute to 410 KPa absolute (1.5 to 4.1 atm absolute).

2. The method as claimed in claim 1, wherein the distilling further comprises:
feeding water into the dividing wall column at a temperature of from 50 to 100° C. into the receiving side of the dividing wall while feeding the crude EO feed stream into the dividing wall column on the receiving side of the dividing wall at a point below a point at which the feeding of the water takes place.

3. The method as claimed in claim 1, wherein the distilling further comprises:
drawing a bottoms stream comprising water and ethylene glycol from a bottom of the dividing wall column and drawing an overhead stream comprising ethylene oxide, gases and condensable vapors from a top of the dividing wall column; and
indirectly heating the dividing wall column by heating at least a portion of the bottoms stream in two separate reboilers, wherein a first reboiler is heated by steam and a second reboiler is optionally heated by a recycled vent gas stream.

4. The method as claimed in claim 3, further comprising:
recycling the overhead stream through a partial condenser to form a reflux liquid stream and a vent gas stream; and
feeding back to the dividing wall column the reflux liquid from the overhead stream.

5. The method as claimed in claim 4, further comprising:
feeding the vent gas stream through a vent gas scrubber; and, then
compressing the scrubbed vent gas stream by either mechanical compression or by passing it through a two-stage jet compressor comprising, from upstream to downstream, a first jet compressor, a partial condenser, and a second jet compressor, to form the recycled vent gas stream that is used as the heating medium in the second reboiler.

6. The method as claimed in claim 1, further comprising:
preheating the crude EO stream to a temperature of from 90 to 140° C. using water from a recycled bottoms stream.

7. The method as claimed in claim 6, wherein the preheating of the crude EO feed stream takes place in a heat exchanger that is directly connected to the receiving side of the dividing wall at a point where the crude EO feed stream enters the dividing wall column.

8. The method as claimed in claim 1, wherein the residence time during which a mixture containing more than 1 wt. % of each of ethylene oxide and water, based on the total weight of the mixture, is present at a temperature above 80° C., is 2 minutes or less.
